(19) United States
(12) Patent Application Publication
(10) Pub. No.: US 2023/0210623 A1
(43) Pub. Date: Jul. 6, 2023

(54) DEVICE FOR PROTECTING MEDICAL OPERATING DEVICES AND/OR EXAMINATION DEVICES SUCH AS AN ENDOSCOPE FROM AERIAL CONTAMINATION

(71) Applicant: ASSISTANCE PUBLIQUE - HOPITAUX DE PARIS, Paris 12 (FR)

(72) Inventor: Stanislas CHAUSSADE, Paris (FR)

(21) Appl. No.: 18/001,156

(22) PCT Filed: Jun. 11, 2021
(86) PCT No.: PCT/EP2021/065857

§ 371 Date: Dec. 8, 2022

(30) Foreign Application Priority Data
Jun. 12, 2020 (FR) FR2006180

(51) Int. Cl. A61B 50/30; B65D 81/20

(52) U.S. Cl. CPC A61B 50/30; B65D 81/2069

(57) ABSTRACT

A device for protecting against an airborne contamination of a medical device including an air circulation cooling system is disclosed. The device also includes an enclosure defining a sealed receiving volume for the medical device, the protection device further comprising structure for circulating gas flow, within the enclosure, the gas flow having predetermined composition and/or quality criteria different from the external environment and able to be used by the cooling system of the medical device in operation.

The present invention relates to a device for protecting against airborne contaminations of surgical and/or examination medical devices, such as an endoscope.

The current COVID-19 epidemia has highlighted the risk of airborne transmission by viruses. SARS-CoV-2, which causes COVID-19, can be transmitted by physical contact from person to person (e.g. handles, kissing) as well as by indirect contact through virus-loaded droplets expelled from an infected person by coughing and sneezing. Although said latter contact mode suggests some transport of the viral load via the indoor air, the droplets (>10 microns) expelled only travel approximately 1 to 2 meters before being deposited on the surrounding surfaces (Heffernan, 2020; REHVA, 2020).

A recent experimental study suggests that the virus could be detected in indoor air as well as on various surfaces a few hours to several hours after their aerosolization in the air and their deposition (van Doremalen et al., 2020). It should be noted here that this aerosolization was carried out using a jet nebulizer (≤ 5 microns) which does not reflect the usual dispersal context (by coughing, sneezing and expectoration). In addition, although the authors report that the viruses were still viable after a few hours, nothing indicates that they had retained their infectious power. It should also be noted that the viral load decreases exponentially with time. However, it should be noted that certain medical activities or interventions administered in care settings (but rarely at home) can generate aerosols likely to contain the virus.

In gastroenterology and more particularly in the context of digestive endoscopy in COVID-19 positive patients, and in particular in intensive care, but also in endoscopy units, the problem of airborne transmission of COVID-19 must be examined. Indeed, all endoscopies that pass into the oropharynx (gastric fibroscopy, endoscopic ultrasound, enteroscopy, endoscopic retrograde cholangiopancreatography known under the acronym ERCP) are considered to be interventions at risk of projecting viral particles for endoscopists, non-medical personnel and the environment, and this for two reasons. The first is that the virus is present in the secretions of the oropharynx; the second is that the endoscopes used in digestive endoscopy comprise (unlike bronchoscopes or endoscopes used in Otorhinolaryngology (ENT) for example) a channel allowing air and water to be blown therethrough. This insufflation, particularly in the oropharynx, is responsible for the aerosolization of viral particles which can circulate in the environment and in the air of the facility where the endoscopy is carried out, whether this is in an intensive care unit, in operating rooms or in an endoscopy unit.

In the context of Covid-19, this transmission is via mucus droplets from the airways, but airborne transmission is not formally excluded. However, this risk of airborne transmission has already been identified for other viruses such as influenza, but also and especially for the SARS virus in 2003 (Evidence of Airborne Transmission of the Severe Acute Respiratory Syndrome Virus.I. Yu, Y Li, T Wai Wong, Wi Tam, AT. Chan, J H.W. Lee, D Y.C. Leung, and T Ho. N Engl J Med 2004;350:1731-9). Publications of that time had even shown the possibility of airborne infection of the virus from one building to another via the ventilation systems from one patient, in airplanes and in hospital facilities (L. Morawska, J. Cao, Airborne transmission of SARS-CoV-2: the world should face the reality, Environment International (2020), doi: https://doi.org/10.1016/j.envint.2020.105730).

Another mode of transmission is by “fomites” which are deposits of viruses on different surfaces of the environment that will be touched by the patient or hospital personnel or which can be remobilized in the air (role of ventilation?). These fomites can, based on the temperature, the humidity and the nature of the surface, remain viable for several hours or even several days (Aerosol and Surface Stability of SARS-CoV-2 as Compared with SARS-CoV-1. N van Doremalen, T Bushmaker, M G. Holbrook, et al. NEJM 2020; DOI: 10.1056/NEJMc2004973).

At the moment, health authorities do not require the prevention of airborne diseases, but it is probable that the current Covid-19 epidemic will change mentalities and raise awareness of the risks associated with the airborne transmission of viruses or other microorganisms, particularly in care facilities. Thus, it is quite necessary to indicate that in the latest recommendations of 2018 by the ESGE, ASGE (USA) or Japan (2019) concerning the disinfection of endoscopes, no mention is made of the transmission of microorganisms by air and no recommendation is made regarding the air circuit (Reprocessing of flexible endoscopes and endoscopic accessories used in gastrointestinal endoscopy: Position Statement of the European Society of Gastrointestinal Endoscopy (ESGE) and European Society of Gastroenterology Nurses and Associates (ESGENA) - Update 2018. U Beilenhoff, H Biering, R Blum, J, M Cimbro, JM Dumonceau, C Hassan, M Jung, B Kampf, C Neumann, M Pietsch, L Pineau, T Ponchon, S Rejchrt, J-F Rey, V Schmidt, J Tillett, J E. van Hooft. Endoscopy 2018; 50: 1205-1234).

During the disinfection of medical devices such as endoscopes, it has been proposed to store these devices in storage cabinets as disclosed in document EP-A-1 290 983. This document thus describes a cabinet used for the hyperaseptic storage of flexible endoscopes. Such a hyperaseptic storage cabinet makes it possible to maintain endoscopes at the level of asepsis obtained after the decontamination/drying phase of the disinfection protocol applied to this type of equipment, in accordance with the regulations in force, until they are next used.

Such cabinets therefore facilitate hyperaseptic storage in order to maintain different items or products at the disinfection level imposed by standards and regulations, based on the nature and destination of said articles or products such as: surgical instrumentation, food products, pharmaceutical products, clothing, etc. Thus, by regulation, endoscopes must be stored in a clean and dry place, away from any source of microbial contamination. Such cabinets are used to store these devices such as endoscopes by keeping them in an aseptic atmosphere prior to their use. To this end, the cabinet comprises a technical compartment in which the air circulates in order to be treated (filtered) and an endoscope storage enclosure in which the filtered air circulates. Such a device thus offers two distinct internal spaces, one being a technical compartment containing the air treatment means and the other being a storage enclosure receiving additional endoscopes.

Documents WO-A-2010/130010, US-A-3683 638 and FR-A-2 935 603 describe similar storage cabinets. Advantageously, such devices facilitate storage at the desired level of asepsis for equipment that has been treated and is awaiting use.

Gastroenterologists were required to carry out digestive endoscopies on COVID-19 positive patients, particularly in intensive care, but also in endoscopy units. At this time, the question of airborne transmission of COVID-19 from biological secretions or virus deposits in the environment of intensive care units and endoscopy units was put forward.

The following observations were made regarding the systems used to carry out digestive endoscopy:
The processors and light generators of endoscopes are systems that have greatly evolved in their design, but technological advances have particularly focused on improving the lens (high-definition), and magnifying or using light filters to better see the vessels and the pit pattern (NBI, ICI, Iscan).
The risk of infection by bacteria is well known for examinations investigating the bile ducts (such as the endoscopic retrograde cholangiopancreatography examination known under the acronym ERCP). It is considered by some as the Achilles’ heel of digestive endoscopy. The decontamination of endoscopes has also benefited from considerable improvement and very strict protocols which have particularly concerned the endoscope itself, the working channel of the endoscope or the air/water channels inside the endoscope, but has not concerned, or hardly concerned, the air and water transmission circuits outside the endoscope. These systems allow air or water to be insufflated during digestive endoscopy. These contaminations, particularly in the context of ERCP, have been responsible for serious bacterial infections and sometimes death. Regarding the risk of viral transmission, the latter is focused on the risk of transmission of the hepatitis B and C viruses where the viral reservoir is in the blood and not in the air.
Contamination from the ambient air in endoscopy rooms or from the endoscopy columns in intensive care units has been examined very little in the literature.
The recent occurrence of the COVID-19 epidemic has relaunched the debate on the risk of transmission by air or by particles deposited in the environment of viral diseases to medical personnel or to another patient because the generators and processors of our endoscopes are used for each endoscopy procedure and are sometimes used in intensive care units and then in endoscopy units for example, which poses the problem of said viral particles being transmitted from one patient to another. In addition, the columns can alternatively be used in Covid- or Covid+ patients.

By focusing then on the air circuit in the sources and the processors used in digestive endoscopy and by opening up a digestive endoscopy processor and a light source, the following has been noted:
All of these devices have an air cooling system by one or more fans. They are never open and they are not cleaned or disinfected. Regardless of the brand, all of these devices have an air intake which comprises one or two grilles to draw the air inside the processor or air source and facilitate a reduction in temperature induced by the light source (xenon, LED lamp, etc.) or the processors. In this way, the temperature in the vicinity of the xenon lamp has been measured at 323° C. The temperature recommended by manufacturers inside these processors should be between 10 and 40° C., which justifies the presence of highly effective ventilation. The temperature in the middle of the processor is close to 25° C. Beyond 40° C., there is a risk of deterioration of the microprocessors. In addition, there is no antimicrobial filter at the inlet or outlet of these processors.
Inside the processor and/or the light source (which may, depending on the model, be in one or two separate enclosures depending on the model and brand of endoscope), the air diffuses widely in order to reduce the temperature at the level of the light unit and the microprocessors. The temperature recordings show that the temperature is around 26° C. and is stable even after extensive use for several hours.
This air is drawn in by one or two fans which discharge the air from inside the processor into the external environment (most often laterally or behind the processor). The distance at which particles can spread is not known but is several meters. The fans used are extremely powerful (for some brand, there are two fans in the light source working at a rate of 1.9 m3/minute that is more than 200 m3 per hour for the light source alone, knowing that the average duration of a digestive endoscopy varies from 30 minutes to 1 hour).
Another element has attracted attention. The air that is used for insufflation through the endoscope is taken from within the enclosure containing the processor without there being any actual filters, by virtue of an air pump connected to the processor-endoscope interface via a plastic tube. In other words, the air injected into the upper or lower digestive tract comes from the external environment, that is from the environment surrounding patients suffering from serious bacterial or viral conditions as the digestive endoscopy requires close contact (< 1 m) between the patient, the physician and the endoscopy system.

For SARS, it has been clearly demonstrated that individuals located less than 1 meter from patients had a very high risk of contamination. When the examination is finished, the fans stop, the endoscope is disconnected from the processor so that it can be decontaminated but when it is turned on for the next patient, the microorganisms potentially present in the processor or the air pump can be ventilated into the external environment and therefore contaminate another patient or caregiver or even, be directly deposited in the digestive tract or the oropharynx during the insufflation required for the endoscope to be inserted into the mouth towards the esophagus. Finally, these air pumps are never checked, never sampled and could be responsible for the growth of bacteria which could be introduced into the human body during insufflation.

Finally, the air taken from the enclosure is also used to instill water into the digestive tract because diverting said air to the water vial makes it possible to increase the pressure above the water level and allows this water to pass through the water channel or air/water channel of the endoscope.

The purpose of the present invention is therefore to overcome these disadvantages by proposing a device for protecting against airborne contaminations of surgical and/or examination medical devices such as an endoscope when they are used in a potentially polluted or contaminated atmosphere.

For this purpose, the invention relates to a device for protecting against an airborne contamination of a medical device comprising an air circulation cooling system, characterized in that it comprises an enclosure defining a volume for receiving said medical device, the protection device further comprising means for circulating gas flow, within the enclosure, this gas flow having predetermined composition and/or quality criteria creating an atmosphere within the enclosure that is different from the external environment and can be used by the cooling system of said medical device in operation.

Thus, the protection device makes it possible to create therein a different atmosphere and preferably isolated from the outside, created by this circulating gas flow, meeting predetermined composition and/or quality criteria in order to comply with hospital hygiene and health and safety rules when this device is used in a medical environment.

Thus, in order to operate, the air cooling system of the processor and of the light source, where appropriate, of the medical device housed in the enclosure and therefore isolated from the external environment, uses the prevailing atmosphere within the enclosure and this cooling system is therefore maintained under the same controlled conditions that meet the health and safety rules imposed.

This thus prevents an airborne contamination of these internal components of the medical device when they are used in a potentially polluted and/or contaminated environment, and prevents the contamination/pollution from spreading in the operating unit or intervention room, during subsequent uses of the medical device.

According to one particularly advantageous embodiment of the device according to the invention, the gas flow circulating in the protective device consists of ambient air originating from the external environment in which the protection device is located. Preferably, the enclosure is furthermore closed in a sealed manner with respect to the external environment.

The circulation means notably comprise:
means for collecting and guiding air from the external environment towards the inner volume of the enclosure, through inlet means arranged on the enclosure,
filtration means for controlling and determining the composition and/or quality of the air before it enters the enclosure,
and means for expelling the air out of the enclosure.

Preferably, the collecting and guiding means consist of suction means such as a motor and an associated fan, positioned outside the enclosure in front of the air inlet means consisting of an air inlet such as an opening arranged in the enclosure and featuring the filtration means. The actuation of the fan causes a pressure drop behind it, creating a continuous flow of air towards the air inlet.

It is also possible to provide air suction means such as a pump connected to the air inlet, the pump being able to enclose the filtration means and/or water cooling systems.

As filtration means, very high efficiency-type filters (HEPA filters) also known as high-efficiency particulate air-type filters are preferably used. HEPA filters represent a highly effective means for filtering dirt, pollen, bacteria and any living particle or microorganism having a size greater than 0.3 microns out of the air. They are used in hospitals, pharmaceutical companies, laboratories and electronic companies, etc. to remove all fine dust. In order to be called HEPA, a filter must remove 99.97% of particles at least 0.3 microns in size.

Suction means such as a pump and filtration means are preferably located outside the inner volume of the enclosure in order to allow easy access to replace the filters, check their operation and carry out maintenance.

In the atmosphere thus created in the enclosure, isolated from the external environment, since the enclosure is kept sealed from the outside, the medical device also isolated from the external environment, is installed. “Sealed” means that the enclosure is designed to allow only the entry of treated external air that meets the desired health criteria, particularly during the operation of the medical device.

A protection device according to the invention thus makes it possible to create an atmosphere within the enclosure, which is different and isolated from the outside environment, thus providing effective protection against an airborne contamination that could exist in this external environment. Advantageously, the prevailing atmosphere in the enclosure is “clean” and can be used for the air circulation cooling system of the medical component without risk of contaminating the latter and by the medical device itself if it uses air to perform an examination or intervention, such as, for example, the system for air insufflation into the digestive tract if the medical device is an endoscope. In this way, only “clean” air, that is having been filtered and therefore decontaminated to meet predetermined composition and/or quality criteria, enters and circulates within the enclosure and can also be used to perform the examination or the intervention.

The air outlet means consist of a one-way valve or non-return valve, provided in a wall of the enclosure, such that the air can only be discharged towards the external environment. The air can also simply be discharged outwards in the operating room as the air used for cooling has been filtered and does not contain microorganisms. However, in order to further guarantee the quality of the air that is released into the external environment, filtration means can also be positioned at the air outlet.

Such circulation means are advantageous, since they are interesting from an economic point of view, the prevailing atmosphere in the enclosure simply consisting of air taken from the external environment but having been filtered in order to meet the required criteria.

According to another very advantageous embodiment, it is also conceivable to make provision that the gas flow circulating in the device comes from a specific gas source such as a source of neutral gas or air that has already undergone treatment or an analysis guaranteeing its characteristics such as air known as medical air. This solution makes it possible to avoid adding an air filtration system outside the device. In this embodiment, the enclosure can also be sealed.

The circulation means notably comprise:
means for connecting a gas flow source such as medical air to the inner volume of the enclosure, arranged on the enclosure, and
means for discharging the gas flow out of the enclosure.

It is then possible to provide a circuit for circulating the gas flow in a closed loop, comprising the source, a pump connected to a pipe guiding the gas flow towards the inlet in the enclosure and an outlet in the enclosure connected to a return pipe towards the source in order to be recycled therein. The gas flow may also simply be discharged outwards in the operating room as the gas flow, such as medical air, does not contain any microorganisms.

In order to enhance the cooling of the processor or any other equipment generating heat in the medical device, the enclosure optionally comprises blowing means for blowing the medical air for example or the filtered outside air entering at its inlet into the enclosure (as in the previous embodiment), through the entire enclosure and notably towards the interior of the medical device, in particular the housing enclosing the processor, such that the air is blown into the entire enclosure and through the medical device to evacuate the heat towards the front of the enclosure where the gas flow outlet means can then be located.

Thus, these blowing means can consist of a blow rail comprising a perforated tube, attached inside the enclosure, for example on its rear face. The front face of the enclosure then preferably has outlet means for the gas flow. The blowing means can thus be connected to the means for connecting to a gas flow source, such as a medical air distribution circuit, as exists in a hospital environment.

The invention therefore proposes a protection device comprising a receptacle or enclosure, preferably made of a synthetic material such as polymethyl methacrylate (PMMA) or any other appropriate material, wherein the medical device comprising a heat-generating component such as a processor, light source or other requiring ventilation cooling is enclosed so as to be isolated from the external environment while being cooled by a “clean” gas supply. The enclosure of the protection device according to the invention is provided with means enabling the operation and adjustment of the medical device contained therein. The walls of the enclosure can notably be rigid or it may be envisaged that the walls are made of a flexible material such as a plastic film, mounted on a rigid frame, in the manner of a cover or a bell.

The medical device generally has a housing containing various components, including a processor, a light source generating heat during operation and also having an air cooling system.

Thus, the enclosure of the protection device according to the invention in which the medical device is housed has appropriate means to enable the operation (such as electrical connections, channels to connect accessories, cables) as well as the adjustment (access to the adjustment buttons) of the medical device contained therein.

In particular, the enclosure has on one or more walls, electrical connection means for connecting the medical device to an electrical power supply, connections between the light source and the processor and means for connecting examination or intervention instruments, for example, such as a flexible endoscope, thus enabling the operation and use of the medical device housed in the protection device.

The enclosure of the device according to the invention is of rectangular parallelepiped shape, defining an inner volume, preferably sealed from the outside, designed to accommodate the housing of the device to be protected. The dimensions of the enclosure make it possible to define a reasonable volume to be housed on the mobile trolleys of the endoscopy columns, for example, but which also has a sufficient volume to allow the renewal of the air contained in the enclosure which will be ventilated in the processor and source. The shape and dimensions of the enclosure are adapted to the shape of the device being required to be housed therein.

The air inlet and outlet are sufficiently sized to allow a sufficient volume of filtered air to enter the enclosure, in particular multiple filters can be used and it is then necessary to have, just like in respirators, a pump for drawing air into the environment and passing it through the filters. Alternatively, the enclosure comprises means for connecting to a medical air source.

It is also possible to provide air cooling means, so that the flow rate of the fans of the cooling means can thus be reduced.

The enclosure comprises an opening enabling access to the inner volume in order to install the device to be protected, this opening having closure means ensuring the hermetic closure of the enclosure relative to the external environment. This opening is provided on one of the faces of the enclosure.

This opening in one face therefore preferably comprises reversible closure means, such as a flap pivotably mounted between a closed position and an open position or a removable or retractable panel, thus enabling the device in the enclosure to always be accessible.

According to a preferred form of the invention, this removable panel forms an entire face of the enclosure, which is then assembled and disassembled from the other faces by force fitting, for example, and comprises sealing means such as a peripheral seal which, once the interlocking is completed, the enclosure, thus assembled, is hermetic, isolating its inner volume from the external environment. Such an opening thus enables the medical device to be positioned and enables access notably to the processor or to the light source in the event of a breakdown.

It is possible to provide, as a variant, that the face of the enclosure consists of a peripheral edge assembled to the other faces and a removable panel, which can be assembled on this peripheral edge in a sealed manner, or pivotably mounted.

According to a preferred embodiment, the front face of the enclosure is configured to enable access and handling of the members for controlling and/or adjusting and/or connecting accessories of the medical device, generally located most commonly on a front face of the device to be protected.

According to a first variant, the front face of the enclosure consists of a wall made of a transparent flexible material, which can have an opening provided with sealing means such as a seal, for the passage and connection of an accessory such as the endoscope on the front face of the housing of the device.

The flexibility of the material forming the front face of the enclosure thus makes it possible to manipulate the control and/or adjustment members such as keys, rotary buttons, etc., and its transparency makes these members visible but also display screens for the operating data which may be present.

According to a preferred variant, the front face may consist of a flexible wall, only one side of which is attached to the rest of the enclosure, the blowing means in the enclosure forcing the medical or filtered air all around the processor and blowing towards the front face which, being flexible, can lift under the effect of the blown air in order to evacuate both the latter and the heat. An overpressure in the enclosure is thus easily avoided. This movable flexible face also enables access to the connection sockets of the endoscope, for example, and the adjustment buttons. In this case, the enclosure enables the medical device to be isolated from the outside, but this enclosure is not completely sealingly closed.

According to another preferred variant of realisation, one of the faces of the enclosure, generally the front face, has an opening intended to house the device to be protected such that part of said device provided with members for adjusting, controlling and/or connecting accessories projects from the enclosure through this opening which, further, has sealing means enabling the sealed engagement of the device to be protected in this opening.

Direct access thus remains to the control face of the device to be protected such as an endoscope, enabling easy handling, while maintaining the seal of the inner volume of the enclosure.

One of the faces of the enclosure, preferably the rear face, is provided with an integrated electrical socket enabling the electrical connection of the medical device, the processor and the light source generally being connected to the 220 V current. Where necessary, one of the faces also enables the connections to be routed between the processor and the light source. The enclosure can also comprise means for measuring the temperature and pressure in order to check the prevailing temperature and pressure inside the enclosure.

Such a protection device can be used to protect equipment, some components (processors, light source, etc.) of which emit heat during operation and require an integrated cooling system and which are used in surgical units or in interventional settings, particularly in radiology, ultrasonography, cardiology, surgery, digestive, bronchial, ENT, urological endoscopy, etc. A medical device is therefore understood to mean any equipment comprising an integrated cooling system used in a medical context and subject to strict hygiene rules. It is conceivable that the protection device can be used in fields other than medical fields imposing composition and/or air quality criteria imposed.

Such components are, in particular, the processor and the light source of medical devices such as flexible or rigid digestive, bronchial, ENT, urological, surgical endoscopes; those of scalpels with a monopolar or bipolar coagulation system, cold light, ultrasound, laser, or cryotherapy devices, radiofrequency devices, operating theater computers of any kind, extracorporeal circulation devices (CPB), microscopes, articulated robots and arms, simulators, drills, radiology and ultrasound apparatus used in the operating room, anesthesia machines with cooling systems, scalpels, surgical laparoscopy system, surgical smoke evacuation system during laparoscopy for example.

In addition, very advantageously, this protection device according to the invention bypasses the need to modify the medical device, such as an endoscope and therefore bypasses the need to modify the current technology of the processors used in endoscopy, as their cooling system is not modified.

It is thus possible to imagine one or more standardized protection devices that can be used in an operating room with variable filtration flow rates depending on the devices to be protected, having a variable size but which fit on the standardized trolleys in order to receive the protection devices but having an adaptable part specific to each equipment.

In the case of an endoscope, the protection device therefore makes it possible to isolate the processor and the light source of flexible or rigid endoscopes in a closed space defined by the enclosure, in order not to draw up living particles into the system and not to discharge them into the external environment or into the endoscope located in the body of the patient.

Thus, an assembly is proposed consisting of the protection device and the medical device, which can be used safely even in a potentially contaminated/polluted environment.

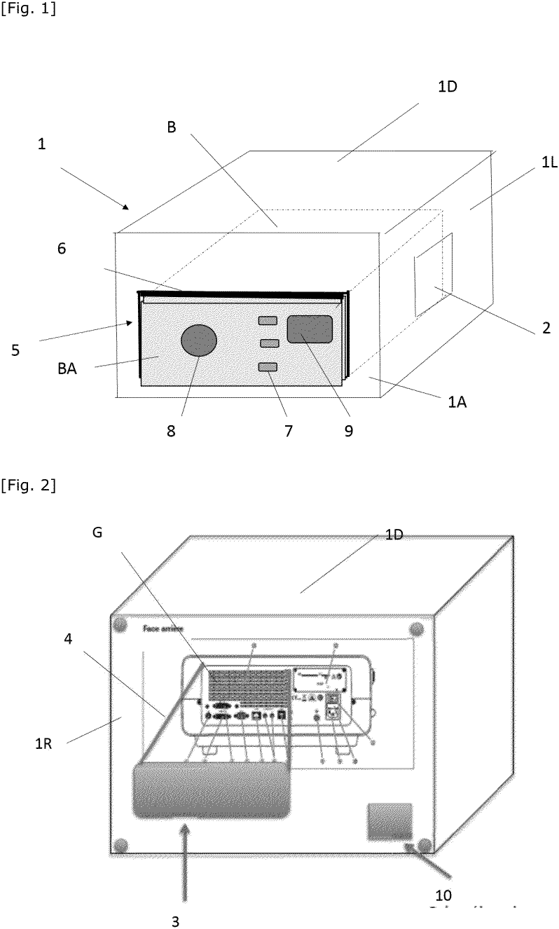

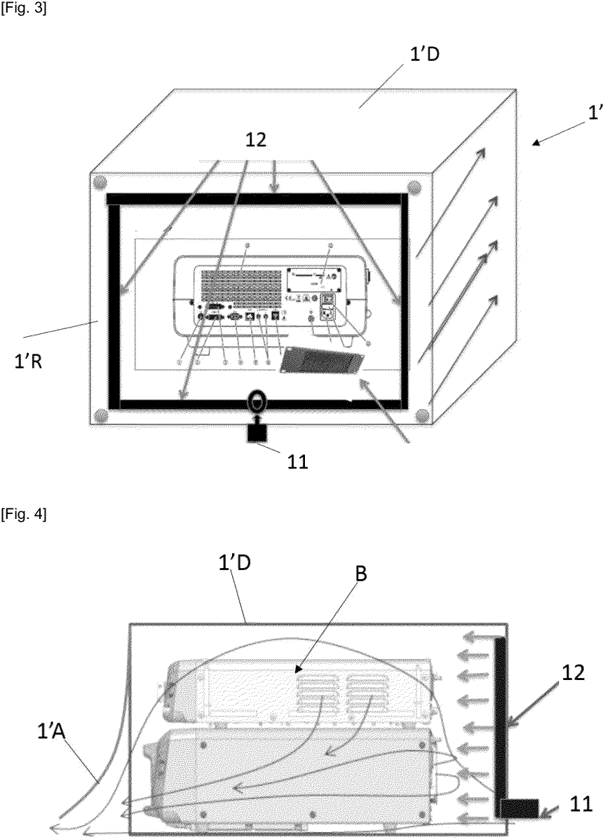

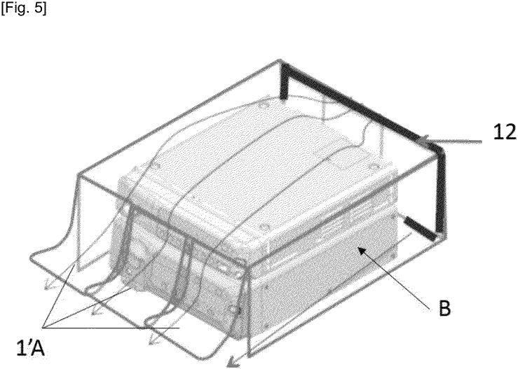

The invention will now be described in greater detail with reference to the drawing in which the figures represent:

[FIG. 1] a front side perspective view of a first embodiment of a protection device according to the invention containing a medical device;

[FIG. 2] a rear side perspective view of the device of FIG. 1;

[FIG. 3] a rear perspective view of a second embodiment of the invention;

[FIG. 4] a side perspective view of FIG. 3; and

[FIG. 5] a perspective top view of the device of FIG. 4.

As can be seen in FIG. 1, a protection device according to the invention comprises an enclosure 1 of rectangular parallelepiped shape. This enclosure 1 defines an inner, preferably sealed, volume in which a medical device such as a digestive endoscope is housed.

These endoscopes generally comprise a housing B having at least one processor and at least one light source such as a xenon lamp, light-emitting diodes (LED), etc. These elements generate heat during operation that must be dissipated. For this purpose, such housing B also comprises an air cooling system with one or more fans. These endoscopes all have, regardless of brand, an air intake which comprises one or two grilles G to draw up the air inside the processor or the air source and enable a temperature reduction induced by the light source or processors and then evacuate the air.

Therefore, inside the processor and/or the light source (which may, depending on the model, be in one or two separate enclosures depending on the models and brand of the endoscope), the air from the atmosphere surrounding the medical device is drawn up by one or two fans, diffused widely in order to reduce the temperature of the light source and microprocessors, and is then discharged from inside the housing B of the device into the external environment.

In addition, the air that is used for insufflation through the endoscope is generally taken from within the housing B containing the processor without there being special provisions for monitoring the quality of this air, by virtue of an air pump connected to the processor-endoscope interface by a plastic tube. In other words, the air injected into the upper or lower digestive tract comes from the external environment, that is to say from the environment of the patients who may suffer from serious bacterial or viral conditions, as digestive endoscopy requires close contact (< 1 m) between the patient, the physician and the endoscopy system.

As already mentioned above, when there is a risk of the external environment being contaminated, there is a risk that the medical device may also be contaminated by the air circulating in the cooling system of said medical device.

In order to avoid this contamination of the medical device and the transmission to other patients or other places, the protection device according to the invention is used. This device comprises an enclosure 1 which, in the example shown, has a generally rectangular parallelepiped shape, the dimensions of which are suitable for defining an inner volume in which the housing B can be installed containing the processor and the light source of a digestive endoscope.

This enclosure 1 is configured to define the volume so that the latter is isolated, for example in a sealed manner from the external environment. Thus, this enclosure 1 has an opening allowing the housing B to be installed in the enclosure 1 but which, once the opening is closed, is hermetically sealed from the external environment.

This enclosure 1 therefore has a bottom face, a top face 1D, a front face 1A, a rear face 1B and two side faces 1L. One of the faces, for example the top face 1D, comprises an opening provided with closing means such as a pivotably mounted flap or a removable or retractable panel. This face 1D can thus itself constitute the means for closing the opening. In this case, this face 1D can be assembled and disassembled from the others by force interlocking for example, and has sealing means such as a peripheral seal enabling, once the interlocking has been carried out, the enclosure 1 thus assembled, to be hermetically sealed, isolating the housing B housed therein from the external environment, thus protecting it from an airborne contamination.

The protection device further comprises means for circulating “clean” air inside the enclosure 1. This enclosure 1 thus comprises, for example, on a side face 1L, an air inlet 2 and on another face, for example the rear face 1B, an air outlet 3, which are associated with the means enabling “clean” air to circulate in the enclosure.

These circulation means can notably comprise air suction means such as a fan actuated by a motor, enabling the air to be drawn towards the inside of the enclosure 1 through the inlet 2. This motor and its associated fan are positioned externally in front of the air inlet 2. The actuation of the fan causes a pressure drop behind it, creating a continuous flow of air towards the air inlet 2. This inlet 2 is then preferably provided with appropriate filtration means to prevent the passage of living microorganisms such as bacteria, viruses, yeast which may be found in the aspirated air. These filters are very high efficiency-type filters (HEPA filters). They thus make it possible to predetermine, through the filtration characteristics, the composition and/or quality of the so-called “clean” air supply, according to health and safety rules.

The suction means and the filtration means are positioned outside the enclosure 1 in order to be easily maintained or replaced. The filters can thus be housed at the air inlet, accessible from the outside.

In this way, only “clean” air, that is having been filtered and therefore decontaminated can enter and circulate in the enclosure 1 according to the predetermined composition and/or quality criteria, that is without contaminants such as viruses, bacteria and microorganisms.

As a result, this “clean” air is the air that will then circulate inside the housing B as cooling air and as air blown through the endoscope.

Preferably, the air outlet 3 arranged for example in the rear face 1R of the enclosure 1 consists of a one-way valve or non-return valve such that the air can only be discharged to the external environment. It is, in particular, possible to provide a pipe 4 between the air vent G of the housing B and the air outlet 3, this latter then also being an air vent. In order to further guarantee the quality of the air that is released into the external environment, filtration means can also be placed at the air outlet 3.

According to a preferred embodiment, the front face 1A of the enclosure 1 is configured to allow access to the control and adjustment buttons 7 of the medical device as well as the connection 8 of the endoscope to the housing B, without needing to open the enclosure 1.

According to the alternative embodiment shown in FIG. 1, the front face 1A has an opening 5 through which the front face BA of the housing B of the medical device can be engaged so as to protrude from the enclosure 1. Furthermore, this opening 5 has sealing means such as a peripheral seal 6 enabling the sealed engagement of the housing B in this opening 5. Direct access thus remains at the front face BA of the housing B enabling easy handling of the control and adjustment members 7, the connection of the endoscope to the connection 8 and the data display screens 9 to be viewed.

One of the faces of the enclosure 1, preferably, the rear face 1B, is provided with an integrated electrical socket 10 enabling the electrical connection of the medical device, with the processor and the light source generally being connected to the 220 V current. This rear face 1B also comprises means for measuring the temperature and pressure to control the temperature and pressure within the enclosure 1.

The protection device according to the invention is therefore an easy to implement and secure solution, the enclosure further being easy to clean.

As can be seen in FIG. 3, the protection device according to a second embodiment of the invention comprises an enclosure 1′ whose rear face 1′R comprises means of connection 11 to a “medical” air source. These connection means 11 guide this medical air into blowing means 12 comprising notably a perforated tube in the form of a rectangular frame arranged on the inner face of the rear face 1′R of the enclosure 1′, which makes it possible to distribute the air forwards throughout the inner volume of the enclosure 1′. This medical air thus enters the housing B, the air vent G of which is located conventionally at the rear of said housing B, towards the cooling circuit of the processor of the medical device and discharges the heat towards the front of the enclosure 1′.

The front face 1′A of the enclosure 1′ consists of a flexible wall such as a flexible plastic sheet, having for example three sections. This wall is attached only to the upper face 1′D of the enclosure 1′ and is free with respect to the other faces or walls. In this way, the air blown towards the front of the enclosure 1′ can be evacuated by lifting this flexible wall. In this case, the enclosure 1′ is no longer totally isolated from the outside, but the circulation of medical air or even of filtered outside air, imposed by the blowing means 12 makes it possible to generate an atmosphere within the enclosure 1′, which is different and isolated from the external environment due to the circulating flow, such that the cooling circuit of the medical device housed in the housing B is thus cooled using a gas flow having predetermined composition and/or quality criteria that differ from the external environment, protecting it from an airborne contamination even when this medical device is used in a place in which the atmosphere may potentially be contaminated.

The enclosure 1′ of the protective device further comprises all appropriate means, passages, connections, enabling the medical device to be connected for its operation.

1. A device for protecting against an airborne contamination of a medical device comprising an air circulation cooling system, comprising: an enclosuredefining a volume for receiving said medical device; the protection device further comprising means for circulating gas flow, within the enclosure; the gas flow having predetermined composition and/or quality criteria creating an atmosphere within the enclosure, which is different from the external environment and can be used by the cooling system of said medical device in operation.

2. The protection device according to the claim 1, characterized in that the enclosure is kept sealed against the external environment.

3. The protection device according to claim 1, characterized in that the gas flow circulating in the protective device consists of ambient air from the external environment in which the protection device is located, the circulation means comprising:
means for collecting and guiding air from the external environment towards the inner volume of the enclosure, through air inlet means arranged on the enclosure;
filtration means for controlling and determining the composition and/or the quality of the air before it enters the enclosure; and
air outlet means outside the enclosure.

4. The protection device according to claim 3, characterized in that the collecting and guiding means consist of suction means such as a motor and an associated fan, positioned outside the enclosure in front of the air inlet means consisting of an air inlet such as an opening arranged in the enclosure, having the filtration means.

5. The protection device according to claim 3, characterized in that the collecting and guiding means consist of suction means such as a pump connected to the air inlet means, the pump being able to contain the filtration means.

6. The protection device according to claim 3, characterized in that the filtration means are very high efficiency-type filters (HEPA filters).

7. The protection device according to claim 3, characterized in that the air outlet means consist of a one-way valve or a non-return valve such that the air can only be discharged to the external environment.

8. The protection device according to claim 7, characterized in that the air outlet means comprise filtration means.

9. The protection device according to claim 3, characterized in that it has a pipe for guiding air between an air vent of the housing and the air outlet.

10. The protection device according to claim 1, characterized in that the gas flow comes from a specific gas source such as a neutral gas source or an air supply having predetermined composition and/or quality criteria, the means for circulating this gas flow comprising means for connecting said source to the inner volume of the enclosure, arranged on the enclosure and
means for discharging the gas flow out of the enclosure.

11. The protection device according to claim 3, characterized in that the enclosure comprises blowing means making it possible to blow the gas flow entering throughout the entire enclosure.

12. The protection device according to claim 10, characterized in that the circulation means form a closed loop gas flow circulation circuit, comprising the source, a pump connected to a pipe guiding the gas flow towards the inlet in the enclosure and an outlet in the enclosure connected to a return pipe towards the source.

13. The protection device according to claim 11, characterized in that the blowing means comprise a blow ramp consisting of a perforated tube, fixed to the inside and to the rear of the enclosure on its rear face.

14. The device according to claim 10, characterized in that the gas flow consists of medical air.

15. The protection device according to claim 1, characterized in that the enclosurein which the medical device is received has appropriate means to enable the operation as well as the adjustment of the medical device contained therein.

16. The protection device according to claim 15, characterized in that the enclosure is of rectangular parallelepiped shape, one of the faces of which has an opening having closure means ensuring the closure of the enclosure relative to the external environment.

17. The protection device according to claim 16, characterized in that the closure means consist of a removable panel constituting one face of the enclosure which can be assembled and disassembled by force interlocking on the enclosure and which has sealing means such as a peripheral seal to enable the enclosure to be hermetically sealed once the interlocking has been carried out.

18. The device according to claim 15, characterized in that the front face of the enclosure consists of a wall made of a transparent flexible material.

19. The device according to claim 15, characterized in that the front face of the enclosure has an opening intended to accommodate the device to be protected, such that part of said device provided with members for adjusting, controlling and/or connecting accessories projects from the enclosure through this opening which has sealing means enabling the sealed engagement of the device to be protected in this opening.

20. The device according to claim 15, characterized in that the front face consists of a flexible wall, only one side of which is attached to the rest of the enclosure, and constituting outlet means for the gas flow.

21. The device according to claim 15, characterized in that the rear face of the enclosure is provided with an integrated electrical socket enabling the electrical connection of the medical device to be protected.

22. The device according to claim 15, characterized in that the enclosure has means for measuring the temperature and the pressure making it possible to control the prevailing temperature and pressure within the enclosure.
